(19) United States
(12) Patent Application Publication
(10) Pub. No.: US 2012/0035597 A1
(43) Pub. Date: Feb. 9, 2012

(54) METHOD FOR TREATING INCISION SURFACES IN A TRANSPARENT MATERIAL

(75) Inventors: Matthias Wottke, Jena (DE); Hartmut Vogelsang, Jena (DE); Wilfried Bissmann, Jena (DE)

(73) Assignee: CARL ZEISS MEDITEC AG, Jena (DE)

(21) Appl. No.: 12/936,382

(22) PCT Filed: Apr. 1, 2009
(86) PCT No.: PCT/EP09/02370

§ 371 Date: Oct. 26, 2011

(30) Foreign Application Priority Data
Apr. 4, 2008 (DE) 102008017771.7
Nov. 6, 2008 (DE) 102008056489.3

(51) Int. Cl. A61F 9/01

(52) U.S. Cl. 606/4

(57) ABSTRACT

A method for the surface treatment (that is, smoothing and/or sealing) of incision surfaces in a transparent material, in particular in the cornea. For this purpose, the incision surface is subjected to the radiation of a laser (for example, UV laser, preferably excimer laser, or IR laser) and/or another suitable radiation source.

### PRIORITY CLAIM

The present application is a National Phase entry of PCT Application No. PCT/EP2009/002370, filed Apr. 1, 2009, which claims priority from German Application Number 10 2008 017771.7, filed Apr. 4, 2008, 2008, and German Application Number 10 2008 056 489.3, filed Nov. 6, 2008, the disclosures of which are hereby incorporated by reference herein in their entirety.

### FIELD OF THE INVENTION

The invention relates to a method for the surface treatment (i.e., smoothing and/or sealing) of cutting planes in a transparent material, particularly in the cornea of the eye.

### BACKGROUND

Cutting planes are formed through producing optical breakthroughs in the material by application of laser radiation focused in the material, wherein the focal point is, for example, shifted three-dimensionally in order to form the cutting planes through sequentially arranged optical breakthroughs. The cutting planes within a transparent material are generated, particularly, in laser-surgical methods, and especially in ophthalmic surgery.

Thereby, the treatment laser radiation within the tissue, i.e. beneath the tissue surface, is focused in such a way that optical breakthroughs in the tissue are formed.

Thereby, several processes initiated by the laser radiation occur in a time sequence in the tissue. If the power density of the radiation exceeds a threshold value, an optical breakthrough will occur, generating a plasma bubble in the material. After the optical breakthrough has been generated, the plasma bubble grows due to expanding gases. If the optical breakthrough is not maintained, the gas generated in the plasma bubble will be absorbed by the surrounding material and the bubble disappears again. However, this process takes very much longer than the forming of the bubble itself. If a plasma is generated at a material boundary, which may also be located within a material structure, material will be removed from the boundary.

This boundary phenomenon is then referred to as photoablation. In connection with a plasma bubble which separates previously connected material layers, the term photodisruption is usually applied. For the sake of simplicity, all such processes herein are collectively termed optical breakthrough, i.e. said term includes not only the actual optical breakthrough, but also the effects resulting therefrom in the material.

For high accuracy of a laser-surgical method, it is valuable to ensure high localization of the effect of the laser beams and preferably avoid collateral damage to adjacent tissue. It is therefore common in prior art to apply the laser radiation in pulsed form, so that the threshold value required for the triggering of an optical breakthrough is exceeded only during the individual pulses for the power density. In this regard, U.S. Pat. No. 5,984,916 clearly shows that the spatial extent of the optical breakthrough (in this case, the generated interaction) strongly depends on the pulse duration.

Therefore, high focusing of the laser beam in combination with very short pulses in the femtosecond range allows for placing of the optical breakthrough in a material with pinpoint accuracy.

The use of pulsed laser radiation has recently become established practice in opthalmology, particularly for laser-surgical correction of defective vision.

Defective vision of the eye often results from the fact that the refractive properties of the cornea and the lens do not effect optimal focusing on the retina.

Aforementioned U.S. Pat. No. 5,984,916 as well as U.S. Pat. No. 6,110,166 describe methods of producing cuts by a suitable generation of optical breakthroughs with the use of fs-lasers, so that, ultimately, the refractive properties of the cornea are specifically influenced. A multitude of optical breakthroughs are sequentially arranged in such a way that a lens-shaped partial volume (lenticule) is isolated within the cornea of the eye. The lens-shaped partial volume, which is separated from the remaining corneal tissue, is then removed from the cornea through a laterally opening cut. The shape of the lenticule is selected in such a way that, after removal, the shape and, thus, the refractive properties of the cornea are modified such that the desired correction of the visual defect is effected. The cutting planes required hereto are curved, which makes a three-dimensional shifting of the focus necessary. Therefore, a two-dimensional deflection of the laser radiation is combined with simultaneous shifting of the focus in a third spatial direction.

In WO 2004/105661, the complete content of which is hereby incorporated by reference, another type of lenticule removal is described, whereby the lenticule is cut by means of the fs-laser in such small fragments that said fragments can be suctioned out by means of one or several cannulae. Therefore, compared to the removal of the entire lenticule, a smaller incision for insertion of the cannula and/or cannulae suffices.

A further application of producing a cut by means of pulsed laser radiation in the cornea is the generation of so-called flaps, i.e., a cut which partially severs a small slice of the cornea in such a way that it can be folded back, making the underlying tissue of an ablation accessible by means of an excimer laser. However, the desired cornea profile is hereby produced through the ablation; the flap is returned to its original position after treatment. Such excimer lasers are known, e.g., from the description of such a laser in U.S. Pat. No. 5,219,344, whereby a large-area ablation is executed with the means of apertures. It is also known how to guide a small-area laser beam from an excimer laser scanningly across the eye and thereby execute a sequential ablation (so-called spot scanning, e.g., DE 19 72 573).

### SUMMARY OF THE INVENTION

The two-dimensional deflection of the laser radiation is, similar to the focus shift, equally important for the accuracy with which the cutting plane can be produced. For the two-dimensional beam guidance, i.e., for the movement of the focus essentially in the plane of the cut, a spiral path is generally traced. It has become apparent that despite the high precision of the beam guidance in the μm range, the cutting planes exhibit a residual roughness, which delay the healing process and also lead to unwanted optical effects.

Even with a three-dimensional shift of the focus and therefore removal of a lenticule, these effects can occur on the cutting planes.

Therefore, the invention is based on designing a method of the aforementioned type in such a way that the generation of a cutting plane with improved quality and faster healing process can be effected.

According to the invention, at least one cutting plane, produced by a femtosecond laser, is exposed to the radiation of a laser (e.g., UV-laser, preferably excimer laser or IR-laser) and/or another suitable radiation source after removal of the partial volume. Suitable are laser beam sources, which lead, e.g., to a necrotization and/or other biochemical or physicochemical changes of the tissue (deactivation, inactivation of cell structures, bondings, etc.), i.e., for example, infrared laser systems (heat build-up, thermal interaction) and/or UV laser systems (photochemical ablation) or other systems, which effect photothermal and/or photochemical processes.

In particular, if smoothing is the priority, in an example embodiment of the invention it is particularly advantageous to moisten the cutting plane before exposure to the radiation source with a liquid, which is, preferably, a sterile saline solution (balanced salt solution BSS). However, other liquids are also suitable, whereby their absorption behavior should be approximated as much as possible to the absorption behavior of the cornea.

For achieving surface sealing, in another example embodiment, other substances or pharmaceuticals, such as mitomycin C, can also be helpful.

The improvement (smoothing, sealing) of the cutting planes produced through femtosecond laser keratomes can be realized by means of a beam source integrated in the femtosecond laser keratome or through an external radiation source.

In a particular example embodiment, the radiation can also be applied by use of a radiation guide with handpiece, which is particularly advantageous when the partial volume is to be removed through a small incision or through destruction and subsequent suctioning.

### BRIEF DESCRIPTION OF THE DRAWINGS

In the following, the invention will be further explained by way of examples with reference to the drawing, wherein:

FIG. 1 is a perspective view of a patient during a laser-surgical treatment with a laser-surgical instrument;

FIG. 2 depicts the focusing of a bundle of rays onto the eye of the patient with the instrument in FIG. 1;

FIG. 3 depicts an exemplary image of the cutting direction;

FIG. 4 depicts a cross section through the cornea with a lenticule;

FIG. 5 depicts a cross section through the cornea after removal of the lenticule;

FIG. 6a, 6b, 6c depict an enlarged segment from FIG. 5;

FIG. 7 depicts a cross section through the cornea after the cut up lenticule has been suctioned off with inserted handpiece.

### DETAILED DESCRIPTION

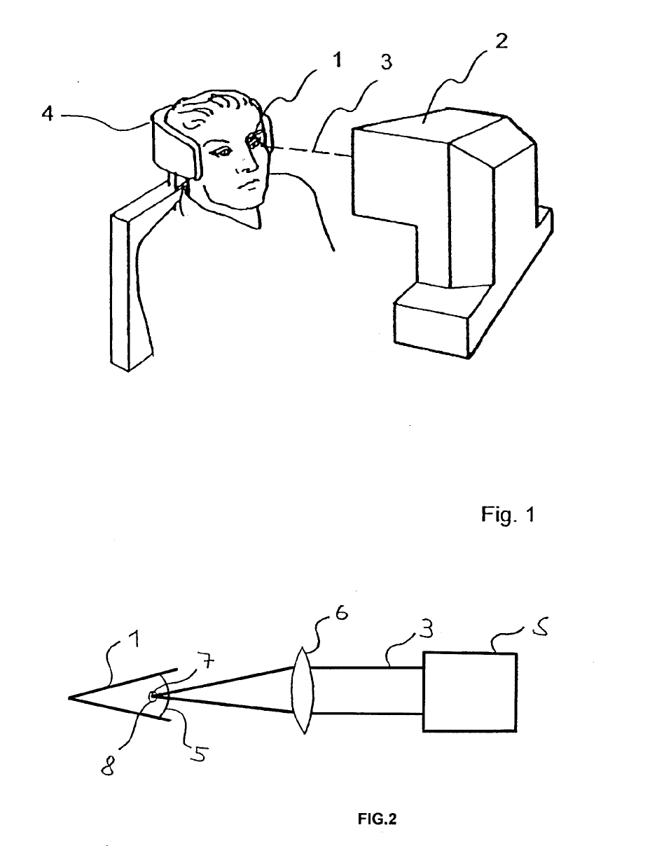

FIG. 1 depicts a laser-surgical instrument for treatment of an eye 1 of a patient, wherein the laser-surgical instrument 2 serves for the execution of a refractive correction. The instrument 2 emits a treatment laser beam 3 onto the eye of the patient 1, whose head is immobilized in a head holder 4. The laser-surgical instrument 2 is capable of generating a pulsed laser beam 3, allowing for the execution of the method described, e.g., in U.S. Pat. No. 6,110,166.

The laser-surgical instrument 2 includes, as schematically shown in FIG. 2, a beam source S, the radiation of which is focused on the cornea 5 of the eye 1. By use of the laser-surgical instrument 2, the defective vision of the eye 1 of the patient can be corrected because material is removed from the cornea 5 in such a way that the refractive properties of the cornea change by a desired degree. Thereby, the material is removed from the stroma of the cornea which lies below the epithelium and Bowman's membrane and above the Decemet's membrane and the endothelium. Alternatively, only one cut in the cornea for the preparation of a flap can be executed with the instrument 2.

The material removal and/or separation is carried out by separating tissue layers through focusing of the high-energy fs-laser beam 3 by a telescope 6 in a focus 7 located in the cornea 5. Thereby, each pulse of the pulsed laser radiation 3 generates an optical breakthrough in the tissue, which initiates a plasma bubble 8.

As a result, the tissue layer separation covers a larger area than the focus 7 of the laser radiation 3. Through suitable deflection of the laser beam 3, many plasma bubbles 8 are now sequentially arranged during treatment. These adjacent plasma bubbles 8 then form a cutting plane 16.

Due to the laser radiation 3, the laser-surgical instrument 2 operates in the manner of a surgical knife which directly separates material layers within the cornea 5 without injuring the surface of the cornea 5. If the cut is guided up to the surface of the cornea 5 (opening cut) by generating further plasma bubbles 8, material of the cornea 5, isolated by the cutting plane 16, can be removed and/or the flap partially lifted and folded back.

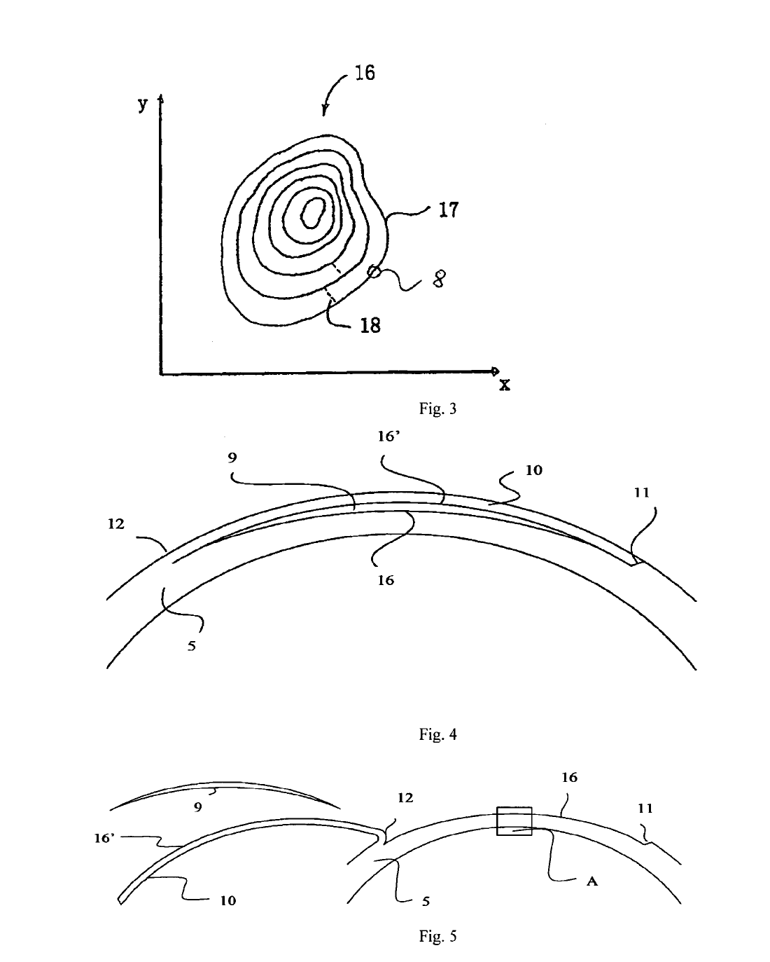

The generation of the cutting plane 16 by the laser-surgical instrument 2 is schematically shown in FIG. 3. The cutting plane 16 is formed by sequential arrangement of the plasma bubbles 8, produced as a result of the continuous shift of the focus 7 of the pulsed focused laser beam 3, along the cutting line 17. In order to remove a partial volume, two such cutting planes 16, 16′ must be created in a suitable geometric arrangement to each other as well as in a suitable form.

The focus shift is effected in an example embodiment by a deflection unit in x and y, not shown in FIG. 2. The telescope 6 is suitably adjusted for a control in the z-direction. As a result, the focus 7 can be adjusted along three orthogonal axes.

For the generation of the cutting plane 16, the focus 7 is now adjusted through the deflection unit in accordance with the cutting lines 17, whereby the zoom optics 6 can, for each cutting line 17, adjust a respective z-coordinate for the focus 7. While the focus 7 passes over a cutting line 17, the telescope 6 can remained fixedly adjusted, and only during the transitions 18, shown as dotted lines in FIG. 3, between the adjacent cutting lines 17 might an adjustment be required.

FIG. 4 depicts a cross section through the cornea 5 as seen after the cutting of two cutting planes 16, 16′ for isolating a lenticule 9. The lenticule 9 is covered by the flap 10, which is confined by an edge cut (opening cut) 11. The edge cut 11 is executed in such a way that part of the corneal tissue is not severed and remains as hinge 12. This ensures that the flap 10 is not entirely severed and therefore easier to reposition.

FIG. 5 shows a cross section through the cornea 5 with opened flap 10; the lenticule 9 has now been removed.

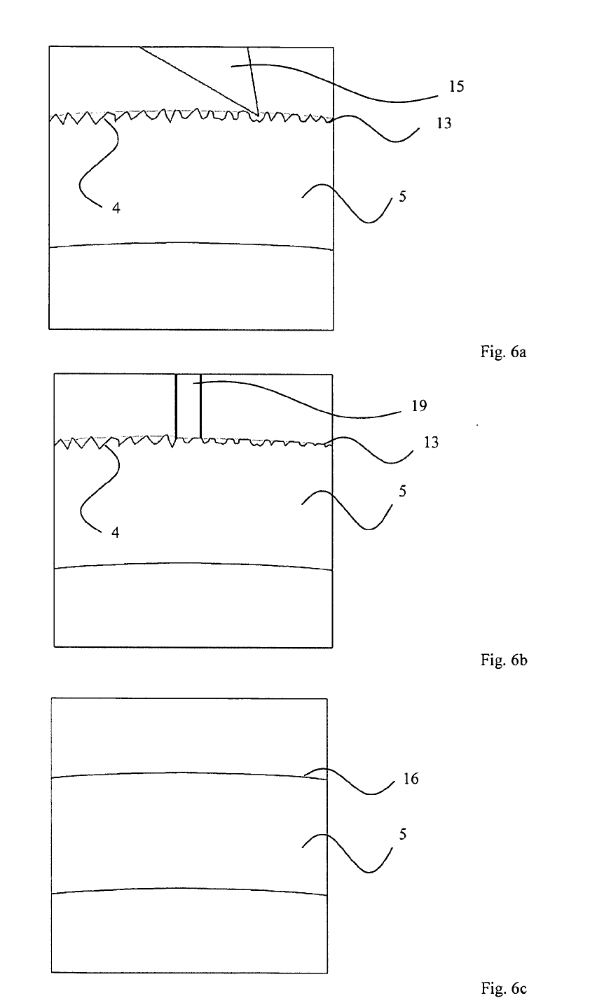

For further clarification of the invention, reference is made to segment A which is shown enlarged in FIG. 6a-6c.

FIG. 6a shows a segment from the cornea 5, wherein the residual roughness 13, resulting from the cutting direction in accordance with FIG. 3, is clearly visible. In order to smoothen said smooth the residual roughness 13 and/or to seal the surface, an example embodiment of the invention also provides for dispensing a liquid 14, preferably a sterile saline solution, by means of a wedge-tipped swab 15 onto the cornea and which, as shown in FIG. 6b, is to be slightly treated with an excimer laser 19. Due to the fact that the liquid 14 exhibits about the same absorption properties and, therefore, ablation properties as the cornea 5 ensures that at first the “tips” of the residual roughness 13 are ablated and no ablation of the actual cornea 5 occurs. As is easily understood, such an approach significantly diminishes the residual roughness 13; the result can be seen in FIG. 6. However, the use of a liquid—as described above—is not stringently required for sealing as well as smoothing.

When liquid is used it is not important whether the excimer laser 19 impinges widespread or in the form of spot scanning on the cornea 5.

In addition, the smoothing/sealing by the excimer laser can, with an additional slight ablation, be combined with further improvement of the optical properties of the eye.

FIG. 6 only shows the smoothing of the lenticule cut 16, but, naturally, the flap cut 16′ can also be appropriately smoothened, whereby said flap cut is positioned on a suitable support and treated with the excimer laser 19.

An infrared laser can also be suitable for the realization of smoothing and/or sealing.

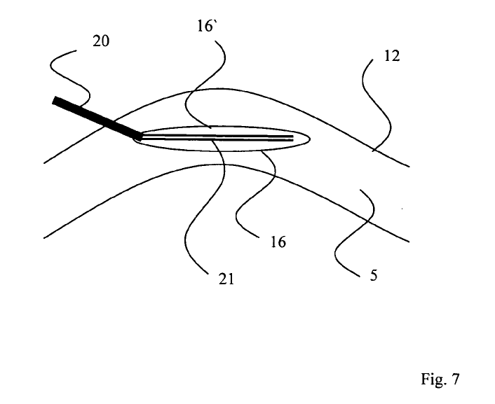

The treatment of flap and lenticule cut can also be executed with the utilization of an appropriate handpiece for guiding the laser radiation into the resulting lenticule cavity without having to fold back the flap. This is shown in FIG. 7. Hereby, a handpiece 20 with a beam area 21, emitting a respective radiation (UV, IR), is inserted in the cavity, which, e.g., develops, according to WO 2004/105661, after suctioning out the lenticule. For guiding the radiation, the handpiece 20 exhibits a radiation guide, not depicted herein. Thereby, it makes sense to irradiate the back of the lenticule 16 as well as the front of the lenticule 16′. This can be achieved either through equipping the handpiece 20 with a second beam area or through insertion with appropriately adjusted orientation.

Through the application of the method, according to the invention, the surface roughness will be diminished, resulting in a faster and optimal healing process.

Thereby, the advantageous effect of the invention can, among others, be based on one or several of the following mechanisms of action:
Necrotizing effect
Apoptosis
Change of the biochemical reactivity
Fibrosis
Metaplasia

1-7. (canceled)

8. A method for the treatment of cutting planes in a cornea of an eye, comprising:
creating cutting planes by application of a femtosecond laser to define and separate a partial volume of the cornea;
removing the partial volume of the cornea; and
applying radiation from an additional radiation source to at least one cutting plane surface.

9. The method according to claim 8, further comprising moistening the cutting plane surface with a liquid before the applying radiation from the additional radiation source.

10. The method according to claim 9, further comprising selecting the liquid to be a sterile saline solution.

11. The method according to claim 10, further comprising selecting the sterile saline solution to be balanced salt solution (BSS).

12. The method according to claim 9, further comprising treating the cutting plane surface with a pharmaceutical before the exposure from the additional radiation source.

13. The method according to claim 8, further comprising integrating the additional radiation source in the femtosecond laser.

14. The method according to claim 8, further comprising operating the additional radiation source separately from the femtosecond laser.

15. The method according to claim 8, further comprising selecting the additional radiation source from a group consisting of an excimer laser, a UV-laser, and an infrared laser.

16. The method according to claim 8, further comprising introducing the additional radiation by use of a handpiece.
